(19) United States
(12) Patent Application Publication
(10) Pub. No.: US 2026/0047991 A1
(43) Pub. Date: Feb. 19, 2026

(54) STIMULI-RESPONSIVE CHROMOGENIC MATERIALS, METHODS OF MAKING AND METHODS OF USE

(71) Applicant: University of Florida Research Foundation, Inc., Gainesville, FL (US)

(72) Inventors: Fan GAO, Gainesville, FL (US); Peng JIANG, Gainesville, FL (US)

(21) Appl. No.: 19/101,641

(22) PCT Filed: Aug. 7, 2023
(86) PCT No.: PCT/US2023/071754

§ 371 Date: Feb. 6, 2025

Related U.S. Application Data
(60) Provisional application No. 63/370,718, filed on Aug. 8, 2022.

(51) Int. Cl. A61K 8/58; A61Q 19/00

(52) U.S. Cl. CPC A61K 8/58; A61Q 19/00; A61K 2800/438; A61K 2800/45; A61K 2800/58; A61K 2800/592

(57) ABSTRACT

In one aspect, the disclosure relates to stimuli-responsive chromogenic compositions that change from a first color to a second color upon application to the skin of a subject and exposure to a stimulus including, but not limited to, heat, light, mechanical rubbing, exposure to electric current, and the like. The compositions include a non-toxic pigment and a cosmetic carrier and do not stain the skin. Additionally, the compositions are stable when stored, even under conditions of elevated temperature. Also disclosed are methods of making the same and methods of treating skin conditions using the same. This abstract is intended as a scanning tool for purposes of searching in the particular art and is not intended to be limiting of the present disclosure.

### CROSS-REFERENCE TO RELATED APPLICATIONS

This application claims the benefit of U.S. Provisional Application No. 63/370,718, filed on Aug. 8, 2022, which is incorporated herein by reference in its entirety.

### BACKGROUND

The global cosmetics market is projected to grow from $380.2 billion in 2019 to $463.5 billion by 2027, with a compound annual growth rate (CAGR) of 5.3% from 2021 to 2027, according to a recent report published by the Allied Market Research. Skin care product sales accounted for $148.3 billion in 2020 and are estimated to be $189.3 billion by 2025. The current emphasis on the development of skin-friendly cosmetic products with smart features has come as a unique opportunity to stand out in the fragmented cosmetics market, and the interests and needs of consumers for the cosmetic industry to develop smart products with stimuli-responsive properties are strong. For instance, although it is directed to use some skincare and cosmetic products in conjunction with an applicator, microcurrent stimulation device, light emitting device, or other stimulus, for a given period of time or until a given amount of exposure to the stimulus is achieved, customers may over- or underestimate stimulus exposure time, thereby not achieving optimal treatment effects.

Disclosed herein are smart chromogenic compositions that change colors in response to the amplitude and duration of external stimuli (e.g., mechanical rubbing, heat, light, etc.). In one aspect, these chromogenic compositions can assist consumers in achieving better treatment effects. Also disclosed herein are chromogenic material systems that exhibit rapid and easily distinguishable color changes in response to a variety of external stimuli, such as mechanical rubbing, brushing, and exposure to electric fields.

### SUMMARY

In accordance with the purpose(s) of the present disclosure, as embodied and broadly described herein, the disclosure, in one aspect, relates to stimuli-responsive chromogenic compositions that change from a first color to a second color upon application to the skin of a subject and exposure to a stimulus including, but not limited to, heat, light, mechanical rubbing, exposure to electric current, and the like. The compositions include a non-toxic pigment and a cosmetic carrier and do not stain the skin. Additionally, the compositions are stable when stored, even under conditions of elevated temperature. Also disclosed are methods of making the same and methods of treating skin conditions using the same.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIGS. 1A-1D show a C174160-based (phthalocyanine blue) gel according to one embodiment of the present disclosure. FIG. 1A: transparent C174160 conductive gel on skin.

FIG. 1B: gel after stimulation with a direct-current electrical device for 30 seconds. FIG. 1C: gel after stimulation with a direct-current electrical device for 2 minutes. FIG. 1D: gel after 10 minutes of skin contact.

FIGS. 2A-2D show a β-carotene-based gel according to one embodiment of the present disclosure. FIG. 2A: transparent β-carotene conductive gel on skin. FIG. 2B: gel after stimulation with a direct-current electrical device for 30 seconds. FIG. 2C: gel after stimulation with a direct-current electrical device for 2 minutes. FIG. 2D: gel after 10 minutes of skin contact.

FIGS. 3A-3D show a Campo® Siddha Sikkappu Thool-based gel according to one embodiment of the present disclosure. FIG. 3A: transparent Campo® Siddha Sikkappu Thool conductive gel on skin. FIG. 3B: gel after stimulation with a direct-current electrical device for 30 seconds. FIG. 3C: gel after stimulation with a direct-current electrical device for 2 minutes. FIG. 3D: gel after 10 minutes of skin contact.

FIGS. 4A-4D show concentration effects of C174160-based (phthalocyanine blue) gel using different weight percentages of C174160, according to one embodiment of the present disclosure. FIG. 4A: transparent C174160 conductive gel on skin. FIG. 4B: gels after stimulation with a direct-current electrical device for 30 seconds. FIG. 4C: gels after stimulation with a direct-current electrical device for 2 minutes. FIG. 4D: gels after 10 minutes of skin contact.

FIGS. 5A-5D show particle size effects of C174160-based (phthalocyanine blue) gel using different particle sizes of C174160, according to one embodiment of the present disclosure. FIG. 5A: transparent C174160 conductive gel on skin. FIG. 5B: gels after stimulation with a direct-current electrical device for 30 seconds. FIG. 5C: gels after stimulation with a direct-current electrical device for 2 minutes. FIG. 5D: gels after 10 minutes of skin contact.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

Disclosed herein are stimuli-responsive chromogenic compositions that include at least one pigment and a cosmetic carrier, wherein the stimuli-responsive chromogenic compositions have a first color prior to application to a skin surface of a subject, and have a second color after application to the skin surface and following exposure to at least one external stimulus.

In an aspect, the at least one pigment is non-toxic. In another aspect, the composition can include from about 0.005 to about 0.04 wt % of the at least one pigment, or about 0.01 wt % of the at least one pigment, or about 0.005, 0.01, 0.015, 0.02, 0.025, 0.03, 0.035, or about 0.04 wt % of the at least one pigment, or a combination of any of the foregoing values, or a range encompassing any of the foregoing values.

In some aspects, the at least one pigment can be phthalocyanine blue (also known as C174160); β-carotene; Campo® Siddha Sikkappu Thool (available from Campo Research, Singapore), also known as Melia azadirachta fruit extract and Coccinia indica flower extract and chemically equivalent to CI14700, or any combination thereof. In one aspect, when the at least one pigment is phthalocyanine blue, the first color can be clear or colorless and the second color can be blue. In another aspect, when the at least one pigment is β-carotene, the first color is light red and the second color is red-orange having a much darker value than the first color. In still another aspect, when the at least one pigment is Campo® Siddha Sikkappu Thool (CL 14700), the first color is clear or colorless and the second color is a light red.

In any of these aspects, the cosmetic carrier can be a soap, cleanser, skin cream, lotion, moisturizer, serum, eye cream, or any combination thereof. In one aspect, the at least one external stimulus can be exposure to an electric field, rubbing, brushing, heat, light, magnetic field, microwave, or any combination thereof. In one aspect, exposure to an electric field can be exposure to a direct current (DC) microcurrent of from about 120 mA to about 375 mA, or of about 120, 125, 150, 175, 200, 225, 250, 275, 300, 325, 350, or about 375 mA, or a combination of any of the foregoing values, or a range encompassing any of the foregoing values.

In any of these aspects, the skin surface can be exposed to the external stimulus for from about 30 seconds to about 10 minutes, or for about 30 seconds, 45 seconds, or 1, 2, 3, 4, 5, 6, 7, 8, 9, or about 10 minutes, or a combination of any of the foregoing values, or a range encompassing any of the foregoing values. In one aspect, the stimuli-responsive chromogenic composition does not stain the skin surface following exposure to the at least one external stimulus.

Also disclosed herein is a process for preparing a stimuli-responsive chromogenic composition, the process including at least the steps of:
(a) suspending at least one pigment powder in a solvent to form a first admixture;
(b) contacting the first admixture with tetraethoxy silane (TEOS) to form a second admixture;
(c) contacting the second admixture with nitric acid to form a suspension;
(d) centrifuging the suspension and discarding a supernatant solution, leaving behind modified particles; and
(e) admixing the modified particles with a carrier to form the stimuli-responsive chromogenic composition.

In an aspect, the first admixture can include about 40 g/L of the at least one pigment powder. In one aspect, prior to performing step (a), the process can further include the step of sieving a pigment precursor powder to obtain at least one pigment powder having a predetermined average particle diameter and/or a narrow particle size distribution. In one aspect, the predetermined average particle diameter can be from about 50 μm to about 1500 μm, or can be about 50, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, or about 1500 μm, or a combination of any of the foregoing values, or a range encompassing any of the foregoing values. In any of these aspects, the at least one pigment powder and the pigment precursor powder are non-toxic.

In one aspect, the at least one pigment powder can be phthalocyanine blue, β-carotene, astaxanthin, or any combination thereof. In another aspect, the solvent can be water, ethanol, isopropanol, acetone, toluene, benzene, or any combination thereof.

In one aspect, in the disclosed process, in step (b), from about 1 g to about 5 g of tetraethoxy silane are added per liter of the first admixture, or about 1, 1.5, 2, 2.5, 3, 3.5, 4, 4.5, or about 5 g of tetraethoxy silane are added per liter of the first admixture, or a combination of any of the foregoing values, or a range encompassing any of the foregoing values. In another aspect, in step (c), from about 2.5 g to about 7.5 g of 0.001 N nitric acid are added per liter of the second admixture, or about 2.5, 3, 3.5, 4, 4.5, 5, 5.5, 6, 6.5, 7, or about 7.5 g of 0.001 N nitric acid are added per liter of the second admixture, or a combination of any of the foregoing values, or a range encompassing any of the foregoing values.

In some aspects, following step (d), the modified particles can be resuspended in the solvent and step (d) can be repeated from about 1 to about 5 times, or 1, 2, 3, 4, or 5 times, or a combination of any of the foregoing values, or a range encompassing any of the foregoing values. In another aspect, the method further includes the step of drying residual solvent from the modified particles prior to performing step (e). In some aspects, the drying can be accomplished in an oven.

In one aspect, when the at least one pigment powder is or includes phthalocyanine blue, the solvent can be ethanol, 3 g of tetraethoxy silane are added to each liter of the first admixture, 6.0 g of 0.001 N nitric acid are added per liter of the second admixture, and the predetermined average particle diameter is from about 50 μm to about 500 μm, or is about 50, 100, 150, 200, 250, 300, 350, 400, 450, or about 500 μm, or a combination of any of the foregoing values, or a range encompassing any of the foregoing values.

In another aspect, when the at least one pigment powder is or includes β-carotene, the solvent can be water, 1.5 g of tetraethoxy silane are added to each liter of the first admixture, 3.0 g of 0.001 N nitric acid are added per liter of the second admixture, and the predetermined average particle diameter is from about 300 μm to about 1500 μm, or is about 300, 350, 400, 450, 500, 550, 600, 650, 700, 750, 800, 850, 900, 950, 1000, 1050, 1100, 1150, 1200, 1250, 1300, 1350, 1400, 1450, or about 1500 μm, or a combination of any of the foregoing values, or a range encompassing any of the foregoing values.

In any of these aspects, the carrier can be a soap, cleanser, skin cream, lotion, moisturizer, serum, eye cream, or any combination thereof. Also disclosed herein are stimuli-responsive chromogenic compositions produced by the disclosed process.

In another aspect, performance of the compositions after a period of storage is identical to performance of the composition immediately after preparation. In one aspect, the period of storage can be from about 1 week to about 6 months, or can be 1, 2, or 3 weeks or 1, 2, 3, 4, 5, or 6 months, or a combination of any of the foregoing values, or a range encompassing any of the foregoing values.

In some aspects, during the period of storage, the composition can be held at an elevated temperature. In one aspect, the elevated temperature can be from about room temperature to about 45° C., or can be about 20, 25, 30, 35, 40, or about 45° C., or a combination of any of the foregoing values, or a range encompassing any of the foregoing values.

Further disclosed herein is a method for treating a skin condition, the method including at least the steps of:
(a) applying the stimuli-responsive chromogenic composition disclosed herein to a skin surface in a subject; and
(b) applying at least one stimulus to the skin surface.

In another aspect, the at least one stimulus can be exposure to an electric field, rubbing, brushing, heat, light, magnetic field, microwave, or any combination thereof. In one aspect, exposure to an electric field can be exposure to a direct current (DC) microcurrent of from about 120 mA to about 375 mA, or of about 120, 125, 150, 175, 200, 225, 250, 275, 300, 325, 350, or about 375 mA, or a combination of any of the foregoing values, or a range encompassing any of the foregoing values. In still another aspect, the at least one stimulus is applied to the skin surface for from about 2 minutes to about 10 minutes. In any of these aspects, the stimuli-responsive chromogenic composition does not stain the skin surface following applying the at least one external stimulus.

In one aspect, the skin condition can be wrinkles, fine lines, dark spots, dryness, oiliness, acne, roughness, dullness, redness, sun damage, loss of elasticity, scarring, hyperpigmentation, rosacea, uneven skin tone, sagging skin, or any combination thereof.

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by,” “comprising,” “comprises,” “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a pigment,” “a stimulus,” or “a cosmetic,” includes, but is not limited to, mixtures or combinations of two or more such pigments, stimuli, or cosmetics, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y.’ The range can also be expressed as an upper limit, e.g. ‘about x, y, z, or less’ and should be interpreted to include the specific ranges of ‘about x,’ ‘about y’, and ‘about z’ as well as the ranges of ‘less than x’, less than y′, and ‘less than z’. Likewise, the phrase ‘about x, y, z, or greater’ should be interpreted to include the specific ranges of ‘about x,’ ‘about y,’ and ‘about z’ as well as the ranges of ‘greater than x,’ greater than y,’ and ‘greater than z.’ In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the term “effective amount” refers to an amount that is sufficient to achieve the desired modification of a physical property of the composition or material. For example, an “effective amount” of a pigment refers to an amount that is sufficient to achieve the desired improvement in the property modulated by the formulation component, e.g. achieving the desired level of color on the skin at specific treatment or stimulation times. The specific level in terms of wt % in a composition required as an effective amount will depend upon a variety of factors including the amount and type of pigment, amount and type of cosmetic composition to which the pigment is added, amount and type of stimulus indicated for treatment, and length of treatment time directed for the cosmetic composition.

As used herein, the terms “optional” or “optionally” means that the subsequently described event or circumstance can or cannot occur, and that the description includes instances where said event or circumstance occurs and instances where it does not.

As used herein, “non-toxic” refers to a compound or composition that does not harm or kill cells or cause any other adverse reaction when administered to a subject such as a human. In one aspect, the stimuli-responsive chromogenic compositions disclosed herein are non-toxic when applied to the skin of a subject.

“Value” as used herein refers to the lightness or darkness of a color. In one aspect, for example, pink can be referred to as a color with a lighter value than a true red. Meanwhile, “color” as used herein refers to a property of an object derived from light interacting with the object and being perceived by photoreceptor cells in the eyes. In some aspects, “color” may be used informally to refer to colorless (clear and/or transparent objects) as well as black, gray, brown, and the like, which do not appear on a color wheel but which are typically understood and referred to by individual consumers as “colors.”

Unless otherwise specified, pressures referred to herein are based on atmospheric pressure (i.e. one atmosphere).

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### ASPECTS

The present disclosure can be described in accordance with the following numbered Aspects, which should not be confused with the claims.
Aspect 1. A stimuli-responsive chromogenic composition comprising at least one pigment and a cosmetic carrier;
wherein the stimuli-responsive chromogenic composition comprises a first color prior to application to a skin surface of a subject; and
wherein the stimuli-responsive chromogenic composition comprises a second color after application to the skin surface and following exposure to at least one external stimulus.
Aspect 2. The stimuli-responsive chromogenic composition of aspect 1, wherein the at least one pigment is non-toxic.
Aspect 3. The stimuli-responsive chromogenic composition of aspect 1 or 2, wherein the composition comprises from about 0.005 to about 0.04 wt % of the at least one pigment.
Aspect 4. The stimuli-responsive chromogenic composition of aspect 1 or 2, wherein the composition comprises about 0.01 wt % of the at least one pigment.
Aspect 5. The stimuli-responsive chromogenic composition any one of aspects 1-4, wherein the at least one pigment comprises phthalocyanine blue, β-carotene, astaxanthin, CI 14700, or any combination thereof.
Aspect 6. The stimuli-responsive chromogenic composition of aspect 5, wherein the at least one pigment is phthalocyanine blue, the first color is clear, and the second color is blue.
Aspect 7. The stimuli-responsive chromogenic composition of aspect 5, wherein the at least one pigment is β-carotene, the first color is light red, and the second color is red-orange having a darker value than the first color.
Aspect 8. The stimuli-responsive chromogenic composition of aspect 5, wherein the at least one pigment is CI 14700, the first color is clear, and the second color is light red.
Aspect 9. The stimuli-responsive chromogenic composition of any one of aspects 1-8, wherein the cosmetic carrier comprises a soap, cleanser, skin cream, lotion, moisturizer, serum, eye cream, or any combination thereof.
Aspect 10. The stimuli-responsive chromogenic composition of any one of aspects 1-9, wherein the at least one external stimulus comprises exposure to an electric field, rubbing, brushing, heat, light, magnetic field, microwave, or any combination thereof.
Aspect 11. The stimuli-responsive chromogenic composition of aspect 10, wherein exposure to an electric field comprises exposure to a direct current (DC) microcurrent of from about 120 mA to about 375 mA.
Aspect 12. The stimuli-responsive chromogenic composition of any one of aspects 1-11, wherein the skin surface is exposed to the at least one external stimulus for from about 30 seconds to about 10 minutes.
Aspect 13. The stimuli-responsive chromogenic composition of any one of aspects 1-12, wherein the stimuli-responsive chromogenic composition does not stain the skin surface following exposure to the at least one external stimulus.
Aspect 14. A process for preparing a stimuli-responsive chromogenic composition, the process comprising:
(a) suspending at least one pigment powder in a solvent to form a first admixture;
(b) contacting the first admixture with tetraethoxy silane to form a second admixture;
(c) contacting the second admixture with nitric acid to form a suspension;
(d) centrifuging the suspension and discarding a supernatant solution, leaving behind modified particles; and
(e) admixing the modified particles with a carrier to form the stimuli-responsive chromogenic composition.
Aspect 15. The process of aspect 14, wherein the first admixture comprises about 40 g/L of the at least one pigment powder.
Aspect 16. The process of aspect 14 or 15, further comprising sieving a pigment precursor powder to obtain at least one pigment powder having a predetermined average particle diameter.
Aspect 17. The process of aspect 16, wherein the predetermined average particle diameter is from about 50 μm to about 1500 μm.
Aspect 18. The process of any one of aspects 14-17, wherein the at least one pigment powder is non-toxic.
Aspect 19. The process of any one of aspects 14-18, wherein the at least one pigment powder comprises phthalocyanine blue, β-carotene, or any combination thereof.
Aspect 20. The process of any one of aspects 14-18, wherein the solvent comprises water, ethanol, isopropanol, acetone, toluene, benzene, or any combination thereof.
Aspect 21. The process of any one of aspects 14-20, wherein, in (b), from about 1 to about 5 g of tetraethoxy silane are added per liter of the first admixture.
Aspect 22. The process of any one of aspects 14-21, wherein, in (c), from about 2.5 to about 7.5 g of 0.001 N nitric acid are added per liter of the second admixture.
Aspect 23. The process of any one of aspects 14-22, wherein, following (d), the modified particles are resuspended in the solvent and step (d) is repeated from 1 to 5 times.
Aspect 24. The process of any one of aspects 14-23, further comprising drying residual solvent from the modified particles prior to performing (e).
Aspect 25. The process of any one of aspects 14-24, wherein the at least one pigment powder comprises phthalocyanine blue, the solvent is ethanol, 3 g of tetraethoxy silane is added to each liter of the first admixture, 6.0 g of 0.001 N nitric acid is added per liter of the second admixture, and the predetermined average particle diameter is from about 50 μm to about 500 μm.
Aspect 26. The process of any one of aspects 14-25, wherein the at least one pigment powder comprises β-carotene, the solvent is water, 1.5 g of tetraethoxy silane is added to each liter of the first admixture, 3.0 g of 0.001 N nitric acid is added per liter of the second admixture, and the predetermined average particle diameter is from about 300 μm to about 1500 μm.
Aspect 27. The process of any one of aspects 14-26, wherein the carrier comprises a soap, cleanser, skin cream, lotion, moisturizer, serum, eye cream, or any combination thereof.
Aspect 28. A stimuli-responsive chromogenic composition produced by the process of any one of aspects 14-27.
Aspect 29. The stimuli-responsive chromogenic composition of any one of aspects 1-13 or 28, wherein performance of the composition after a period of storage is identical to performance of the composition immediately after preparation.
Aspect 30. The stimuli-responsive chromogenic composition of aspect 29, wherein the period of storage is from about 1 week to about 6 months.
Aspect 31. The stimuli-responsive chromogenic composition of aspect 30, wherein, during the period of storage, the composition is held at an elevated temperature.
Aspect 32. The stimuli-responsive chromogenic composition of aspect 31, wherein the elevated temperature is from about room temperature to about 45° C.
Aspect 33. A method for treating a skin condition, the method comprising:
(a) applying the stimuli-responsive chromogenic composition of any one of aspects 1-13 or 28-32 to a skin surface in a subject; and
(b) applying at least one stimulus to the skin surface.
Aspect 34. The method of aspect 33, wherein the at least one stimulus comprises exposure to an electric field, rubbing, brushing, heat, light, or any combination thereof.
Aspect 35. The method of aspect 34, wherein exposure to an electric field comprises exposure to a direct current (DC) microcurrent of from about 120 mA to about 375 mA.
Aspect 36. The method of any one of aspects 33-35, wherein the at least one stimulus is applied to the skin surface for from about 2 minutes to about 10 minutes.
Aspect 37. The method of any one of aspects 33-36, wherein the stimuli-responsive chromogenic composition does not stain the skin surface following applying the at least one external stimulus.
Aspect 38. The method of any one of aspects 33-37, wherein the skin condition comprises wrinkles, fine lines, dark spots, dryness, oiliness, acne, roughness, dullness, redness, sun damage, loss of elasticity, scarring, hyperpigmentation, rosacea, uneven skin tone, sagging skin, or any combination thereof.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1: General Methods

### Surface Modification Methods

Prior to the surface treatment step, commercial pigment powders are sieved to get particles with relatively uniform sizes and relatively narrow size distribution. Exemplary size distributions for different pigments are provided in Example 2. The sieved particles are then suspended in an appropriate solvent such as water or 100% ethanol with a typical concentration of 40 g/L. Under continuous stirring, tetraethoxy silane (TEOS) is dropwisely added to 1 L suspension, followed by adding 0.001 N aqueous nitric acid solution. The amount of TEOS used with different pigments can vary and exemplary volumes are provided in Example 2. The mixture is stirred at room temperature for one hour. The suspension is then centrifuged at 1000 rpm for 5 minutes and the supernatant solution is discarded. The same solvent is added to suspend the surface-modified particles. This centrifugation step is then repeated 5 times. The final traces of solvent can be removed from the particles in an oven. Alternatively, the particles in a trace amount of solvent can be directly used in preparing the particle dispersions for use in the chromogenic compositions disclosed herein.

### Exemplary Stimuli

Several commercial products were used as testing media to demonstrate the chromogenic effects of the disclosed materials, including a conductive gel and the LUMISPA® cleanser from NUSKIN® Enterprises, Inc. (Provo, UT). A Galvanic Spa System and an AGELOC® BOOST™ device from NUSKIN® Enterprises, Inc. were used in providing mechanical rubbing and small DC microcurrents (from 120 mA to 375 mA) to stimulate color changes.

### Example 2: Chromogenic Material Systems

### C174160-Based System

In the first material system, an inexpensive and biosafe cosmetic pigment-C174160 (phthalocyanine blue, 29H,31H-phthalocyaninato (2-)-N29,N30,N31,N32) copper), which has been widely used in cosmetic products (e.g., soap bars and facial care products), is first treated using the disclosed surface modification technology and then dispersed in the NUSKIN® conductive gel or the AGELOC® LUMISPAR cleanser (both available from NUSKIN® Enterprises, Inc., Provo, UT).

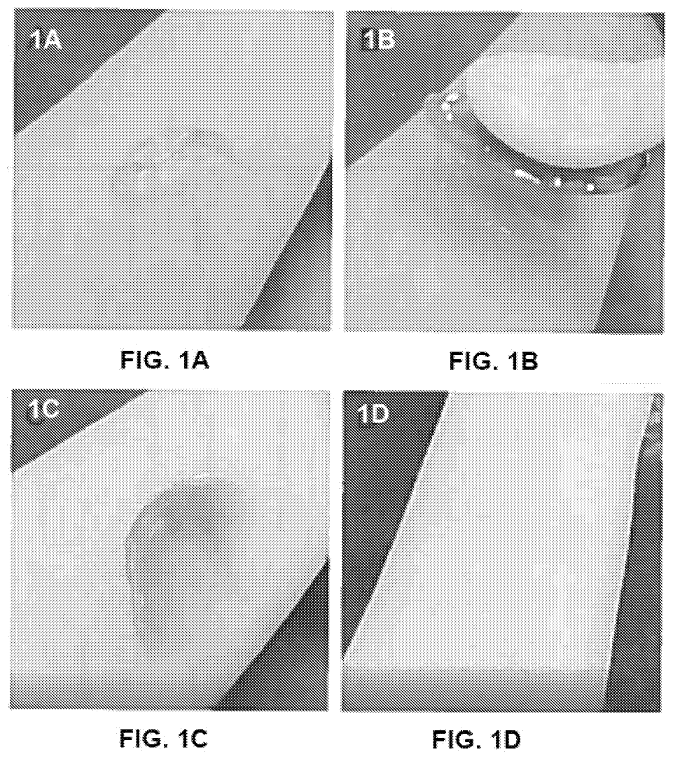

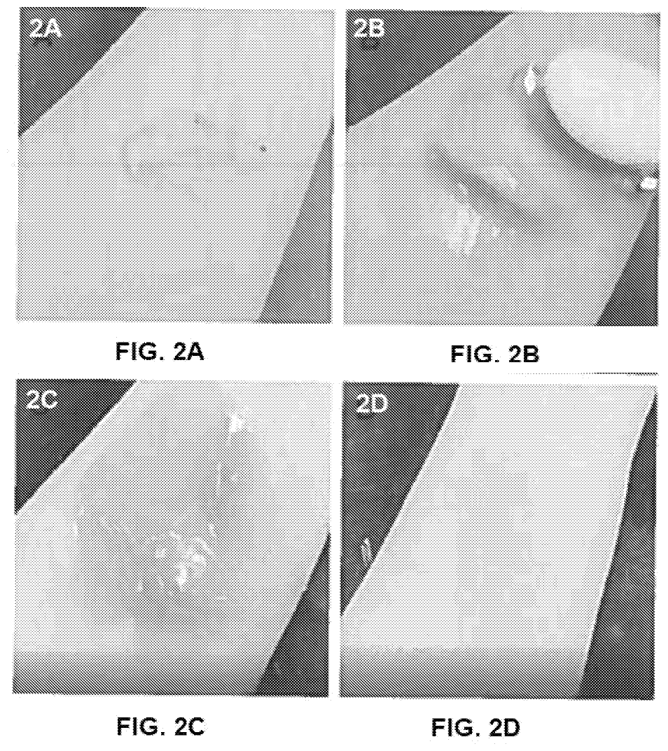

The concentration of the C174160 particles can be adjusted, with the optimal concentration being 0.01 wt. %. The gel/dye mixture rapidly changes color from transparent (FIG. 1A) to an easily distinguishable blue color (FIG. 1B) after turning on the microcurrent device and massaging the gel that is in contact with the skin. The gel color can change to dark blue after operating for less than two minutes (FIG. 1C). Importantly, the skin is not dyed even after 10-minute contact with the resulting dark blue gel (FIG. 1D). Furthermore, the modified C174160/gel mixture exhibits superior durability and shelf life. Prepared samples show nearly identical chromatic responses even after accelerated aging tests at 45° C. for 4 weeks followed by storage at room temperature for more than 6 months.

β-carotene-Based System

Similar to the C174160 material system, another dye that works well with existing commercial products such as the NUSKIN® conductive gel is β-carotene, which is a safe food pigment extracted from carrots and other plants with vibrant red/orange colors. (β-carotene can be converted into vitamin A (retinol) and is considered an important antioxidant in the body. It helps boost the skin's health by increasing the skin's defenses against UV radiation.

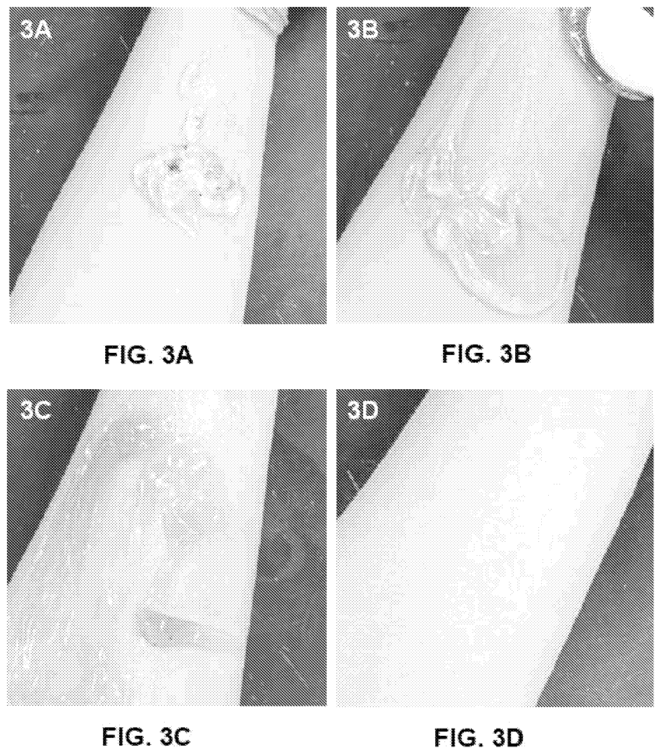

It was further discovered that (β-carotene particles after the disclosed treatment in the gel can change color from a little bit red (due to the presence of large undissolved dye particles, see FIG. 2A) to an easily perceptible orange/red color in about 45 seconds using AGELOC® BOOST™ device from NUSKIN® Enterprises, Inc. (FIG. 2B). The gel retains the orange/red color after 2 minutes of treatment (FIG. 2C). The skin is not dyed even after 10-minute contact with the red gel (FIG. 2D). Accelerated aging tests on this new material system were also performed by heating the sample in an oven at 45° C. for one week. The sample was stable and maintains the same chromatic performance as a freshly prepared gel.

### CI14700-Based System

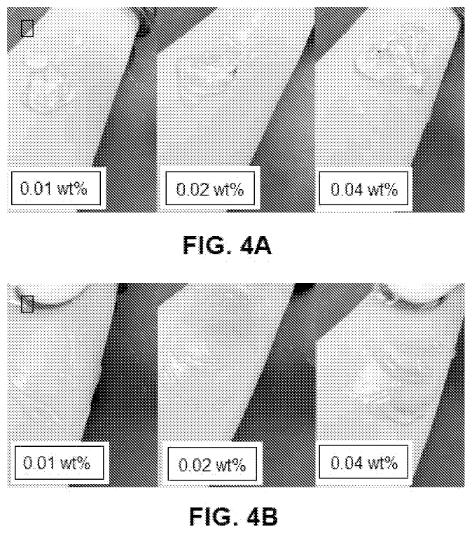

Another dye that works well in the methods disclosed herein is Campo® Siddha Sikkappu Thool (chemically identical to CI 14700). FIG. 3A shows transparent Campo® Siddha Sikkappu Thool conductive gel on skin. FIG. 3B shows the gel after stimulation with a direct-current electrical device for 30 seconds. FIG. 3C shows the gel after stimulation with a direct-current electrical device for 2 minutes. FIG. 3D shows the gel after 10 minutes of skin contact.

### Exemplary Parameters for Surface Modification

Exemplary parameters for surface modification using phthalocyanine blue and β-carotene are provided in Table 1 below:

TABLE 1

| Chromogenic Material | Phthalocyanine blue | β-carotene |
|---|---|---|
| Average Particle Sizes | 50, 100, 200, and/or | 300, 500, 700, and/or |
| | 500 μm | 1500 μm |
| Solvent | 100% Ethanol | Water |
| Amount of TEOS per 1 L | 3 g | 1.5 g |
| Suspension | | |
| Amount of 0.001N | 6.0 g | 3.0 g |
| Aqueous Nitric Acid | | |
| per 1 L Suspension | | |
| Wt % Surface-Modified | 0.005-0.04 | 0.005-0.04 |
| Particles in Suspension | | |

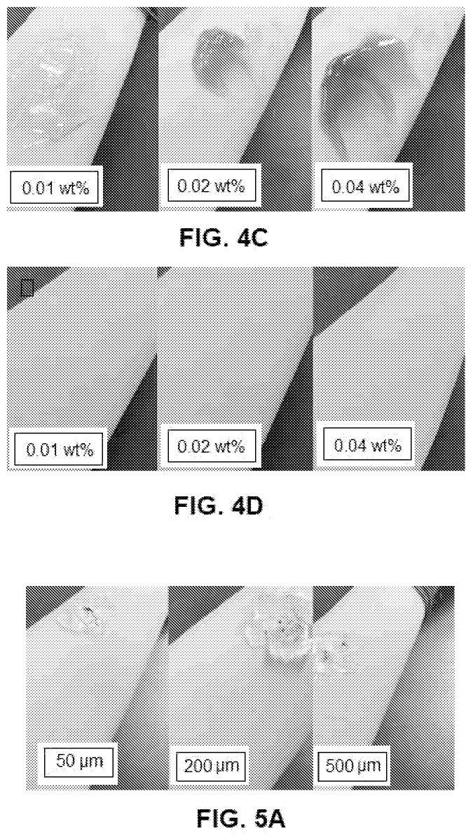

FIGS. 4A-4D show concentration effects of C174160-based (phthalocyanine blue) gel using different weight percentages of C174160, according to one embodiment of the present disclosure. FIG. 4A: transparent C174160 conductive gel on skin. FIG. 4B: gels after stimulation with a direct-current electrical device for 30 seconds. FIG. 4C: gels after stimulation with a direct-current electrical device for 2 minutes. FIG. 4D: gels after 10 minutes of skin contact.

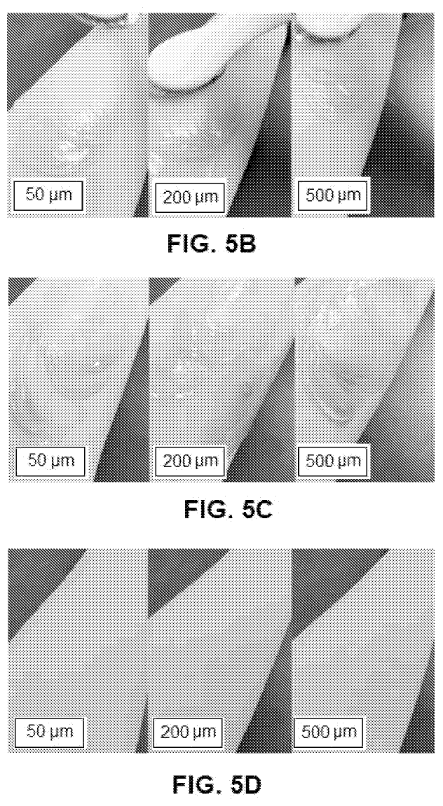

FIGS. 5A-5D show particle size effects of C174160-based (phthalocyanine blue) gel using different particle sizes of C174160, according to one embodiment of the present disclosure. FIG. 5A: transparent C174160 conductive gel on skin. FIG. 5B: gels after stimulation with a direct-current electrical device for 30 seconds. FIG. 5C: gels after stimulation with a direct-current electrical device for 2 minutes. FIG. 5D: gels after 10 minutes of skin contact.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

1. A stimuli-responsive chromogenic composition comprising at least one pigment and a cosmetic carrier;
wherein the stimuli-responsive chromogenic composition comprises a first color prior to application to a skin surface of a subject; and
wherein the stimuli-responsive chromogenic composition comprises a second color after application to the skin surface and following exposure to at least one external stimulus.

2. (canceled)

3. The stimuli-responsive chromogenic composition of claim 1, wherein the composition comprises from about 0.005 to about 0.04 wt % of the at least one pigment.

4. (canceled)

5. The stimuli-responsive chromogenic composition claim 1, wherein the at least one pigment comprises phthalocyanine blue, β-carotene, astaxanthin, CI 14700, or any combination thereof.

6. The stimuli-responsive chromogenic composition of claim 5, wherein the at least one pigment is phthalocyanine blue, the first color is clear, and the second color is blue.

7. The stimuli-responsive chromogenic composition of claim 5, wherein the at least one pigment is β-carotene, the first color is light red, and the second color is red-orange having a darker value than the first color.

8. The stimuli-responsive chromogenic composition of claim 5, wherein the at least one pigment is CI 14700, the first color is clear, and the second color is light red.

9. The stimuli-responsive chromogenic composition of claim 1, wherein the cosmetic carrier comprises a soap, cleanser, skin cream, lotion, moisturizer, serum, eye cream, or any combination thereof.

10. The stimuli-responsive chromogenic composition of claim 1, wherein the at least one external stimulus comprises exposure to an electric field, rubbing, brushing, heat, light, magnetic field, microwave, or any combination thereof.

11. The stimuli-responsive chromogenic composition of claim 10, wherein exposure to an electric field comprises exposure to a direct current (DC) microcurrent of from about 120 mA to about 375 mA.

12. The stimuli-responsive chromogenic composition of claim 1, wherein the skin surface is exposed to the at least one external stimulus for from about 30 seconds to about 10 minutes.

13. The stimuli-responsive chromogenic composition of claim 1, wherein the stimuli-responsive chromogenic composition does not stain the skin surface following exposure to the at least one external stimulus.

14.-28. (canceled)

29. The stimuli-responsive chromogenic composition of claim 1, wherein performance of the composition after a period of storage is identical to performance of the composition immediately after preparation.

30. The stimuli-responsive chromogenic composition of claim 29, wherein the period of storage is from about 1 week to about 6 months.

31. The stimuli-responsive chromogenic composition of claim 30, wherein, during the period of storage, the composition is held at an elevated temperature of from about room temperature to about 45° C.

32. (canceled)

33. A method for treating a skin condition, the method comprising:
(a) applying the stimuli-responsive chromogenic composition of claim 1 to a skin surface in a subject; and
(b) applying at least one stimulus to the skin surface.

34. The method of claim 33, wherein the at least one stimulus comprises exposure to an electric field, rubbing, brushing, heat, light, or any combination thereof.

35. The method of claim 34, wherein exposure to an electric field comprises exposure to a direct current (DC) microcurrent of from about 120 mA to about 375 mA.

36. The method of claim 33, wherein the at least one stimulus is applied to the skin surface for from about 2 minutes to about 10 minutes.

37. The method of claim 33, wherein the stimuli-responsive chromogenic composition does not stain the skin surface following applying the at least one external stimulus.

38. The method of claim 33, wherein the skin condition comprises wrinkles, fine lines, dark spots, dryness, oiliness, acne, roughness, dullness, redness, sun damage, loss of elasticity, scarring, hyperpigmentation, rosacea, uneven skin tone, sagging skin, or any combination thereof.
